(19) United States
(12) Patent Application Publication
(10) Pub. No.: US 2025/0210152 A1
(43) Pub. Date: Jun. 26, 2025

(54) VECTOR GENERATION METHOD AND APPARATUS, DATA PROCESSING METHOD AND APPARATUS, AND STORAGE MEDIUM

(71) Applicant: Lemon Inc., Grand Cayman (KY)

(72) Inventors: Xiang GAO, Los Angeles, CA (US); Weihao GAO, Beijing (CN); Wenzhi XIAO, Beijing (CN); Zhirui WANG, Los Angeles, CA (US); Liang XIANG, Beijing (CN); Chong WANG, Los Angeles, CA (US)

(21) Appl. No.: 18/851,600

(22) PCT Filed: Apr. 11, 2023
(86) PCT No.: PCT/SG2023/050244

§ 371 Date: Sep. 26, 2024

(30) Foreign Application Priority Data
Apr. 12, 2022 (CN) 202210379369.2

(51) Int. Cl. G16C 20/50; G16C 20/70

(52) U.S. Cl. CPC G16C 20/50; G16C 20/70

(57) ABSTRACT

The present disclosure provides a vector generation method, a data processing method, a vector generation apparatus, a data processing apparatus, and a non-transitory computer-readable storage medium. The vector generation method includes: performing learnable processing on N current position vectors in a mapping table to obtain N learnable position vectors, where N is a positive integer; and updating the N current position vectors with the N learnable position vectors.

The present application claims priority to Chinese Patent Application No. 202210379369.2, filed on Apr. 12, 2022, which is incorporated herein by reference in its entirety as a part of the present application.

### TECHNICAL FIELD

Embodiments of the present disclosure relate to a vector generation method, a data processing method, a vector generation apparatus, a data processing apparatus, and a non-transitory computer-readable storage medium.

### BACKGROUND

In the fields of new drug research and development, new material design, energy, and the like, molecular property prediction is a very important technical demand. Molecular property prediction allows for the discovery of a molecule with desired properties, and has important applications in various fields. Potential new drugs or new materials can be defined fast only by accurately and efficiently predicting key properties of a molecule. One kind of method for molecular property prediction is density functional theory (DFT) or another method based on a first principle such as quantum mechanics. Although a molecular shape may be accurately predicted using this method, this method leads to high computational costs. Another kind of method for molecular property prediction is a semi-empirical method, which is high in computing speed but low in accuracy.

### SUMMARY

This section is provided to give a brief overview of concepts, which will be described in detail later in the section Detailed Description. This section is not intended to identify key or necessary features of the claimed technical solutions, nor is it intended to be used to limit the scope of the claimed technical solutions.

At least one embodiment of the present disclosure provides a vector generation method. The method includes: performing learnable processing on N current position vectors in a mapping table to obtain N learnable position vectors, where N is a positive integer; and updating the N current position vectors with the N learnable position vectors.

At least one embodiment of the present disclosure further provides a data processing method. The method includes: obtaining a position parameter to be processed; determining, based on a mapping table corresponding to the position parameter to be processed, a position vector to be processed that corresponds to the position parameter to be processed, where the mapping table includes N current position vectors, and the N current position vectors in the mapping table are obtained through updating based on the N learnable position vectors obtained according to the vector generation method described in any one of the embodiments of the present disclosure; and processing, by using a neural network corresponding to the mapping table, the position vector to be processed to obtain a processing result.

At least one embodiment of the present disclosure further provides a vector generation apparatus. The apparatus includes: one or more memories storing computer-executable instructions in a non-transitory manner; and one or more processors configured to run the computer-executable instructions, where when the computer-executable instructions are run by the one or more processors, the vector generation method according to any one of the embodiments of the present disclosure is implemented.

At least one embodiment of the present disclosure further provides a data processing apparatus. The apparatus includes: one or more memories storing computer-executable instructions in a non-transitory manner; and one or more processors configured to run the computer-executable instructions, where when the computer-executable instructions are run by the one or more processors, the data processing method according to any one of the embodiments of the present disclosure is implemented.

At least one embodiment of the present disclosure further provides a non-transitory computer-readable storage medium. The non-transitory computer-readable storage medium stores computer-executable instructions, where when the computer-executable instructions are executed by a processor, the vector generation method according to any one of the embodiments of the present disclosure is implemented, or the data processing method according to any one of the embodiments of the present disclosure is implemented.

### BRIEF DESCRIPTION OF DRAWINGS

The foregoing and other features, advantages, and aspects of the embodiments of the present disclosure become more apparent with reference to the following specific implementations and in conjunction with the accompanying drawings. Throughout the accompanying drawings, the same or similar reference numerals denote the same or similar elements. It should be understood that the accompanying drawings are schematic and that parts and elements are not necessarily drawn to scale.

FIG. 1A is a schematic flowchart of a vector generation method according to at least one embodiment of the present disclosure;

FIG. 1B is a schematic flowchart of another vector generation method according to at least one embodiment of the present disclosure;

FIG. 2A to FIG. 2D are schematic diagrams of a curve formed by a plurality of current position vectors according to some embodiments of the present disclosure;

FIG. 3A is a schematic diagram of a curve formed by a plurality of learnable position vectors according to some embodiments of the present disclosure;

FIG. 3B is a schematic diagram of a curve formed by a plurality of learnable position vectors according to some embodiments of the present disclosure;

FIG. 3C is a schematic diagram of the plurality of learnable position vectors, shown in FIG. 3A, mapped to a two-dimensional space;

FIG. 3D is a schematic diagram of the plurality of learnable position vectors, shown in FIG. 3B, mapped to a two-dimensional space;

FIG. 4 is a schematic flowchart of a data processing method according to at least one embodiment of the present disclosure;

FIG. 5 is a schematic diagram of a vector generation apparatus according to at least one embodiment of the present disclosure;

FIG. 6 is a schematic diagram of a data processing apparatus according to at least one embodiment of the present disclosure;

FIG. 7 is a schematic diagram of a non-transitory computer-readable storage medium according to at least one embodiment of the present disclosure; and

FIG. 8 is a schematic diagram of a hardware structure of an electronic device according to at least one embodiment of the present disclosure.

### DETAILED DESCRIPTION

The embodiments of the present disclosure are described in more detail below with reference to the accompanying drawings. Although some embodiments of the present disclosure are shown in the accompanying drawings, it should be understood that the present disclosure may be implemented in various forms and should not be construed as being limited to the embodiments set forth herein. Rather, these embodiments are provided for a more thorough and complete understanding of the present disclosure. It should be understood that the accompanying drawings and the embodiments of the present disclosure are only for exemplary purposes, and are not intended to limit the scope of protection of the present disclosure.

It should be understood that steps described in method implementations of the present disclosure may be performed in different orders, and/or performed in parallel. Furthermore, additional steps may be included and/or performance of the illustrated steps may be omitted in the method implementations. The scope of the present disclosure is not limited in this respect.

The term “include/comprise” used herein and the variations thereof are an open-ended inclusion, namely, “include/comprise but not limited to”. The term “based on” is “at least partially based on”. The term “an embodiment” means “at least one embodiment”. The term “another embodiment” means “at least one another embodiment”. The term “some embodiments” means “at least some embodiments”. Related definitions of the other terms will be given in the description below.

It should be noted that the concepts such as “first” and “second” mentioned in the present disclosure are only used to distinguish between different apparatuses, modules, or units, and are not used to limit the sequence or interdependence of functions performed by these apparatuses, modules, or units.

It should be noted that the modifiers “a/an” and “more” mentioned in the present disclosure are illustrative rather than restrictive, and those skilled in the art should understand that the modifiers should be understood as “one or more”, unless the context explicitly dictates otherwise.

The names of messages or information exchanged between a plurality of apparatuses in the implementations of the present disclosure are used for illustrative purposes only, and are not used to limit the scope of these messages or information.

Research has found that in recent years, a machine-learning-based molecular property prediction model is an effective alternative solution to balance accuracy and efficiency. Position information (for example, a distance between atoms and a bond angle) is one of the most basic physical quantities of the machine-learning-based molecular property prediction model.

Currently, as an input to a machine learning model, position information of a molecule is mainly represented by using the following three methods. The first is directly using position information in the form of an original scalar as the input to the machine learning model. However, the form of the original scalar is not conducive for the machine learning model to deal with a complex physical relationship in the molecule. Many physical quantities of the molecule are nonlinear, non-monotonic, and complex with respect to the position information, and therefore the input to the machine learning model is various nonlinear transformations of the position information in an ideal condition. The second is customizing a transformation manner by a user to transform the position information, for example, a reciprocal of the distance between atoms. Such a representation form of the position information may be more effective than directly using the form of the original scalar. However, such transformation is not so universal. For example, the reciprocal of the distance between atoms may be suitable for some potential energy relationships in a polynomial form, but may not necessarily be suitable for other function forms (for example, an exponential function). In addition, such a method is highly dependent on a choice of the user, and thus is limited in universality. The third is transforming the position information using a set of manually selected basis functions (for example, a Gaussian function or a Bessel function). However, this method is also limited in generalization and universality, and the efficiency is determined by the selection of the basis functions.

At least one embodiment of the present disclosure provides a vector generation method. The vector generation method includes: performing learnable processing on N current position vectors in a mapping table to obtain N learnable position vectors, where N is a positive integer; and updating the N current position vectors with the N learnable position vectors.

In the vector generation method provided in this embodiment of the present disclosure, the learnable position vector is obtained through learning. The learnable position vector is not limited to a specific form determined by a selected or customized function, and thus is more universal. The current position vector updated based on the learnable position vector can better simulate complex non-linearity in molecular property prediction to improve accuracy of predicting various molecular properties.

At least one embodiment of the present disclosure further provides a data processing method, a vector generation apparatus, a data processing apparatus, and a non-transitory computer-readable storage medium.

The embodiments of the present disclosure are described in detail below with reference to the accompanying drawings. However, the present disclosure is not limited to these specific embodiments. Detailed description of some known functions and known components is omitted in the present disclosure, to make the following description of the embodiments of the present disclosure clear and concise.

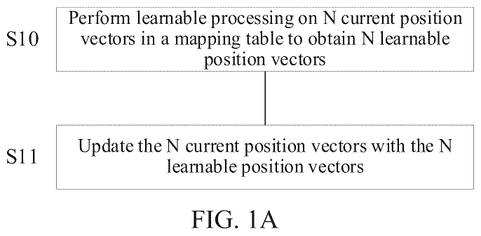

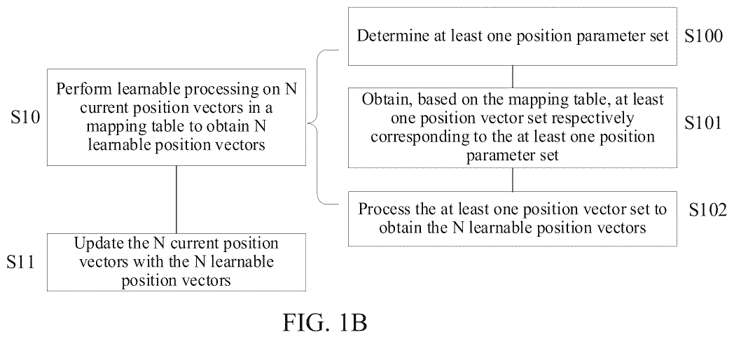

FIG. 1A is a schematic flowchart of a vector generation method according to at least one embodiment of the present disclosure. FIG. 1B is a schematic flowchart of another vector generation method according to at least one embodiment of the present disclosure.

As shown in FIG. 1A, in some embodiments of the present disclosure, the vector generation method may include the following steps S10 and S11.

In step S10, learnable processing is performed on N current position vectors in a mapping table to obtain N learnable position vectors. For example, N is a positive integer, and N is greater than or equal to 1. It should be noted that in the following description of the present disclosure, an example in which N is greater than 1 is used for description, unless otherwise specified.

In step S11, the N current position vectors are updated with the N learnable position vectors.

For example, in some embodiments, the vector generation method further includes: performing the learnable processing on N updated current position vectors again.

In the vector generation method provided in this embodiment of the present disclosure, the learnable processing may be performed multiple times on the current position vectors in the mapping table, and the current position vectors in the mapping table are updated based on the learnable position vectors. Then, the learnable processing may be performed on the updated current position vectors again, until the updated current position vectors meet a preset requirement, so that a final mapping table is obtained. The final mapping table may be used for subsequent processing, for example, used for molecular property prediction.

For example, the mapping table further includes N position parameters in a one-to-one correspondence with the N current position vectors. The mapping table is used to indicate a mapping relationship between a position parameter and a current position vector.

For example, the N current position vectors are in a one-to-one correspondence with the N learnable position vectors.

For example, in some embodiments, as shown in the following Table 1, the N position parameters included in the mapping table are a position parameter d1, a position parameter d2, a position parameter d3, . . . , and a position parameter dN, and the N current position vectors included in the mapping table are a current position vector iv1 corresponding to the position parameter d1, a current position vector iv2 corresponding to the position parameter d2, a current position vector iv3 corresponding to the position parameter d3, . . . , and a current position vector ivN corresponding to the position parameter dN.

TABLE 1

| Position parameter | Current position vector |
|---|---|
| d1 | iv1 |
| d2 | iv2 |
| d3 | iv3 |
| . . . | . . . |
| dN | ivN |

It should be noted that in Table 1, the mapping table is presented in the form of a table, but the present disclosure is not limited thereto. The mapping table may be presented in any form, and this is not specifically limited in the embodiments of the present disclosure.

For example, in some embodiments, a value of N may be set based on a specific situation. For example, N may range from 10 to 1000. For example, N may be 100 or 360.

For example, when the final mapping table is used for molecular property prediction, there is a non-monotonic relationship between a prediction error and N. An excessively large or small N may result in a great prediction error. Therefore, the value of N needs to be selected based on an actual situation.

For example, in some embodiments, the position parameter d1 may be 1 nanometer, the position parameter d2 may be 2 nanometers, the position parameter d3 may be 3 nanometers, and so on. A difference between any two adjacent position parameters in the N position parameters may be 1 nanometer. For example, in some other embodiments, the position parameter d1 may be 1 degree, the position parameter d2 may be 2 degrees, the position parameter d3 may be 3 degrees, and so on. A difference between any two adjacent position parameters in the N position parameters may be 1 degree.

It should be noted that in this embodiment of the present disclosure, two adjacent position parameters mean that there is not any other position parameter in the mapping table between the two position parameters; that is, any one of a plurality of position parameters in the mapping table is not located between the two adjacent position parameters. For example, the position parameter d1 and the position parameter d2 are two adjacent position parameters, and the position parameter d2 and the position parameter d3 are two adjacent position parameters; the position parameter d1 and the position parameter d3 are two nonadjacent position parameters, and the position parameter d2 is between the position parameter d1 and the position parameter d3.

It should be noted that in some embodiments, all of the plurality of position parameters in the mapping table may be integers. However, the present disclosure is not limited thereto, and at least some position parameters in the mapping table may include an integral portion and a decimal part.

For example, in an initial state, a value of each current position vector in the mapping table is a random value; that is, in the initial state, each current position vector in the mapping table is randomly set. Then, the learnable processing is performed on the current position vector by using the vector generation method provided in this embodiment of the present disclosure, to obtain each learnable position vector through learning. Since the learnable position vector is obtained based on learning, the learnable position vector is not limited to a specific form and can better show a complex physical relationship in a molecule.

For example, in some examples, in the initial state, the current position vector iv1 in Table 1 is [0.8, 0, 0.5, 0.8], the current position vector iv2 in Table 1 is [0.8, 0.8, −0.2, −0.1], the current position vector iv3 in Table 1 is [0, 0.1, 0.2, 0.4], and the position vector ivN in Table 1 is [0, 0.5, 0.1, 0.3].

For example, a dimensionality of each current position vector may be set based on the actual situation. For example, in some examples, the dimensionality of each current position vector may range from 32 to 128. For example, in the above example, the dimensionality of each current position vector may be 4; that is, each current position vector includes four elements. For example, for the current position vector iv3=[0, 0.1, 0.2, 0.4], an element of the current position vector iv3 in a first dimension is 0, an element of the current position vector iv3 in a second dimension is 0.1, an element of the current position vector iv3 in a third dimension is 0.2, and an element of the current position vector iv3 in a fourth dimension is 0.4.

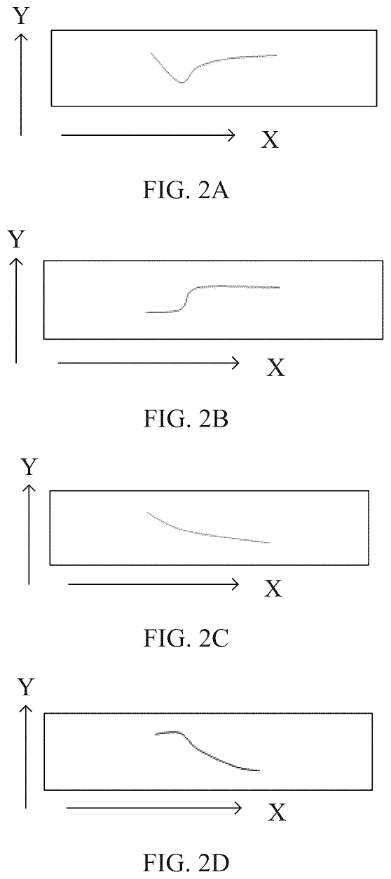

FIG. 2A to FIG. 2D are schematic diagrams of a curve formed by a plurality of current position vectors according to some embodiments of the present disclosure. For example, in FIG. 2A to FIG. 2D, an X direction represents a position parameter (for example, a normalized value), and a Y direction represents a value of an element in each dimension.

In the example shown in FIG. 2A to FIG. 2D, the dimensionality of each current position vector is 4; that is, each current position vector includes four elements. FIG. 2A shows a curve formed by values of elements of the plurality of current position vectors in the first dimension. FIG. 2B shows a curve formed by values of elements of the plurality of current position vectors in the second dimension. FIG. 2C shows a curve formed by values of elements of the plurality of current position vectors in the third dimension. FIG. 2D shows a curve formed by values of elements of the plurality of current position vectors in the fourth dimension.

For example, as shown in FIG. 2A to FIG. 2D, the curve formed by the values of the elements of the plurality of current position vectors in each dimension is a smooth curve.

For example, the dimensionality of each current position vector in the mapping table is fixed; that is, a plurality of dimensions corresponding to each of the plurality of current position vectors in the mapping table are the same.

For example, in some embodiments, as shown in FIG. 1B, the learnable processing in step S10 includes the following steps S100 to S102.

In step S100, at least one position parameter set is determined.

In step S101, at least one position vector set respectively corresponding to the at least one position parameter set is obtained based on the mapping table.

In step S102, the at least one position vector set is processed to obtain the N learnable position vectors.

For example, in step S100, each position parameter set includes at least one of the N position parameters in the mapping table.

For example, in step S100, a number of the at least one position parameter set may be determined based on the actual situation. For example, in some embodiments, the number of the at least one position parameter set may be 32, 48, 64, etc. When the number of the at least one position parameter set is greater than 1, numbers of position parameters respectively corresponding to the plurality of position parameter sets may be the same or may be different. For example, one of the at least one position parameter set may include four position parameters, while another of the at least one position parameter set may include five position parameters. It should be noted that a number of position parameters corresponding to each position parameter set is a number of position parameters included in the position parameter set, and the number of position parameters included in each position parameter set is determined based on the actual situation.

For example, in some embodiments, step S100 includes: determining at least one piece of data to be processed; and determining, based on the at least one piece of data to be processed, the at least one position parameter set in a one-to-one correspondence with the at least one piece of data to be processed.

For example, each piece of data to be processed corresponds to one molecule. The molecule may be a small molecule, a macromolecule, or the like.

For example, when the data to be processed corresponds to a molecule, each position parameter in the mapping table includes a spacing between two adjacent atoms in the molecule and/or a bond angle in the molecule.

For example, the number of position parameters in each position parameter set is determined based on data to be processed that corresponds to the position parameter set. For example, in some embodiments, the position parameter is the spacing between the two adjacent atoms in the molecule. For example, in an example, a molecule may include three atoms: a first atom, a second atom, and a third atom. A spacing between the first atom and the second atom is a first spacing. A spacing between the first atom and the third atom is a second spacing. A spacing between the second atom and the third atom is a third spacing. If the first spacing, the second spacing, and the third spacing are all equal, a position parameter set corresponding to the molecule includes only one position parameter, and the position parameter is the first spacing (or the second spacing or the third spacing); or if the first spacing, the second spacing, and the third spacing are not equal to one another, the position parameter set corresponding to the molecule includes three position parameters, and the three position parameters are the first spacing, the second spacing, and the third spacing.

For example, in some embodiments, step S101 may include: for each position parameter (referred to as a selected position parameter hereinafter) in each position parameter set: in response to the selected position parameter being different from any one of the N position parameters in the mapping table: determining a first position parameter to be interpolated and a second position parameter to be interpolated that correspond to the selected position parameter; determining, based on the mapping table, a current position vector corresponding to the first position parameter to be interpolated as a first position vector to be interpolated and a current position vector corresponding to the second position parameter to be interpolated as a second position vector to be interpolated; and performing interpolation on the first position vector to be interpolated and the second position vector to be interpolated to obtain a current position vector corresponding to the selected position parameter; or in response to the selected position parameter being one of the N position parameters: determining a current position vector corresponding to the selected position parameter directly based on the mapping table.

For example, the first position parameter to be interpolated and the second position parameter to be interpolated are two of the N position parameters in the mapping table, and the selected position parameter is located between the first position parameter to be interpolated and the second position parameter to be interpolated.

For example, an interpolation method corresponding to the interpolation includes linear interpolation and/or cubic Hermite interpolation.

For example, a derivative of a property of the molecule with respect to the position parameter needs to be found in some cases. For example, an atomic force can be obtained by finding a derivative of potential energy of the molecule with respect to the position parameter. In this case, it is required that a vector function corresponding to the current position vector is derivable with respect to the position parameter x; that is, there is g=dh(x)/dx, where x represents the position parameter, h(x) represents the vector function corresponding to the current position vector, and the vector function may be obtained through fitting based on the position parameter and the current position vector in the mapping table. This requirement affects the selection of the interpolation method corresponding to the above-mentioned interpolation. If it is not required that the vector function corresponding to the current position vector is derivable with respect to the position parameter x, the interpolation method corresponding to the interpolation may be linear interpolation. However, linear interpolation may result in the absence of g for a plurality of position parameters in the mapping table. Therefore, linear interpolation cannot be applied to a scenario that requires the property of the molecule to be derivable with respect to the position parameter. If it is required that the vector function corresponding to the current position vector is derivable with respect to the position parameter x, the interpolation method corresponding to the interpolation may be a higher-order interpolation method such as cubic Hermite interpolation. For example, cubic Hermite interpolation uses a higher-order multinomial coefficient, so that the vector function corresponding to the current position vector is derivable with respect to the position parameter.

For example, the vector function corresponding to the current position vector is continuous, and the vector function corresponding to the current position vector is derivable with respect to the position parameter; that is, the vector function corresponding to the current position vector is continuous and derivable. Therefore, infinite position information may be represented by finite learnable position parameters. This can help a neural network better handle complex intramolecular physical interactions and improve model accuracy.

For example, in some embodiments, when the interpolation method corresponding to the interpolation is linear interpolation, the current position vector corresponding to the selected position parameter is expressed as:

$$h ( x ) = ( 1 - t ) * h ( ⌊ x ⌋ ) + t * h ( ⌈ x ⌉ ) ,$$

where h(*) represents the vector function corresponding to the current position vector, x represents the selected position parameter, t represents a weight, └x┘ represents a largest position parameter less than the selected position parameter x in the N position parameters in the mapping table, and ┌x┐ represents a smallest position parameter greater than the selected position parameter x in the N position parameters in the mapping table. For example, in some embodiments, N is 100, and the N position parameters in the mapping table are 1 nanometer, 2 nanometers, 3 nanometers, . . . , and 100 nanometers. When x is 1.3, └x┘ is 1 nanometer, and ┌x┐ is 2 nanometers.

For example, in some embodiments, t may be expressed as:

$$t = \frac{x - ⌊ x ⌋}{⌈ x ⌉ - x} .$$

It should be noted that t may be specifically set based on the actual situation. This is not limited in this embodiment of the present disclosure.

For the mapping table shown in Table 1, the position parameter d1 may be 1 nanometer, the position parameter d2 may be 2 nanometers, the position parameter d3 may be 3 nanometers, and by analogy, the position parameter dN may be 100 nanometers. If the selected position parameter is the position parameter d1, the current position vector iv1 is used as the current position vector corresponding to the selected position parameter. If the selected position parameter is 1.3, the first position parameter to be interpolated and the second position parameter to be interpolated that correspond to the selected position parameter may be determined. For example, the first position parameter to be interpolated is the position parameter d1, and the second position parameter to be interpolated is the position parameter d2. Then, the current position vector iv1 is used as the first position vector to be interpolated, and the current position vector iv2 is used as the second position vector to be interpolated. The interpolation is performed on the current position vector iv1 and the current position vector iv2 to determine the current position vector corresponding to the selected position parameter.

For example, in some embodiments, when the interpolation method corresponding to the interpolation is linear interpolation, weighted summation may be performed on the current position vector iv1 and the current position vector iv2 to obtain the current position vector corresponding to the selected position parameter. For example, a weight corresponding to the current position vector iv1 and a weight corresponding to the current position vector iv2 may be determined by the selected position parameter, the first position parameter to be interpolated, and the second position parameter to be interpolated. For example, in some embodiments, when the selected position parameter is 1.3, the weight corresponding to the current position vector iv1 may be 0.7, and the weight corresponding to the current position vector iv2 may be 0.3. For example, iv1=[0.8, 0, 0.5, 0.8], and iv2=[0.7, 0.8, −0.2, −0.1]. In this case, the current position vector corresponding to the selected position parameter may be expressed as h=70%*iv1+30%*iv2=0.7*[0.8, 0, 0.5, 0.8]+0.3*[0.7, 0.8, −0.2, −0.1].

It should be noted that the weight corresponding to the current position vector iv1 and the weight corresponding to the current position vector iv2 may be set based on the actual situation. This is not limited in this embodiment of the present disclosure. For example, the weight corresponding to the current position vector iv1 and the weight corresponding to the current position vector iv2 may be fixed values, for example, are both 0.5.

For example, in some embodiments, when the interpolation method corresponding to the interpolation is cubic Hermite interpolation, the current position vector corresponding to the selected position parameter is expressed as:

$$h ( x ) = c 1 * h ( ⌊ x ⌋ ) + c 2 * h ( ⌈ x ⌉ ) + c 3 * g ( ⌊ x ⌋ ) + c 4 * g ( ⌈ x ⌉ ) ,$$

where h(*) represents the vector function corresponding to the current position vector, g(*) represents the derivative of the vector function corresponding to the current position vector with respect to the position parameter, that is, g(x)=dh(x)/dx, x represents the selected position parameter, └x┘ represents a largest position parameter less than the selected position parameter x in the N position parameters in the mapping table, ┌x┐ represents a smallest position parameter greater than the selected position parameter x in the N position parameters in the mapping table, and c1 to c4 represent interpolation coefficients, and are respectively expressed as: c1=2*t3−3*t2+1, c2=1−c1, c3=t3−2*t2+1, and c4=t3−t2, where t represents the weight. In this case, h(x) is continuous and derivable.

For example, in step S101, each position vector set includes a current position vector that is obtained based on the mapping table and that corresponds to a position parameter in a position parameter set corresponding to the position vector set.

For example, each position vector set includes at least one current position vector. For example, at least one position parameter in each position parameter set is in a one-to-one correspondence with at least one current position vector in a position vector set corresponding to the position parameter set.

For example, in some embodiments, step S102 may include: separately processing the at least one position vector set using the neural network to obtain at least one output result respectively corresponding to the at least one position vector set; calculating at least one loss value using a loss function corresponding to the neural network based on the mapping table, the at least one position parameter set, and the at least one output result; and in response to the at least one loss value not satisfying a predetermined condition, correcting the N current position vectors based on the at least one loss value to obtain the N learnable position vectors; or in response to the at least one loss value satisfying the predetermined condition, using the N current position vectors as the N learnable position vectors.

In the present disclosure, the learnable processing is implemented by using the neural network. To be specific, the learnable processing is performed on the N current position vectors in the mapping table by using the neural network to obtain an output from the neural network. Then, the N current position vectors are corrected based on the output from the neural network to obtain the N learnable position vectors. In this case, the N current position vectors are used as an input to the neural network. In this embodiment of the present disclosure, the input to the neural network is corrected and updated based on the output learned by the neural network, to finally obtain the mapping table that meets a requirement.

The learnable position vector generated by using the vector generation method provided in this embodiment of the present disclosure is obtained through continuous training and learning based on a machine learning model (for example, the neural network). The current position vector in the mapping table obtained through updating based on the learnable position vector is also obtained through learning based on the machine learning model. Therefore, the current position vector is not limited to a specific form determined by a selected or customized function and is more universal. Complex non-linearity in molecular property prediction can be better simulated based on the current position vector. In addition, when the machine learning model is used to perform molecular prediction based on the current position vector, accuracy of the machine learning model and accuracy of predicting various properties of the molecule using the machine learning model can be improved, and interpretability of the machine learning model can be improved; that is, specific visualization and interpretability can be physically provided.

For example, the vector generation method provided in this embodiment of the present disclosure is not highly dependent on the selection of a specific neural network model. In other words, in step S102, the neural network may have various architectures as long as the neural network uses the position parameter (the spacing between the atoms, the bond angle, or the like) as the input. In actual applications, when the neural network is used for molecular property prediction, the input (that is, the position parameter) to the neural network may be replaced with the current position vector obtained through updating based on the learnable position vector in the present disclosure, to implement molecular property prediction using the neural network. Such a replacement can usually help the neural network handle the complex intramolecular physical interactions and improve the model accuracy.

For example, in some embodiments, the neural network may be a convolutional neural network or a graph neural network.

For example, the predetermined condition may be set based on the actual situation. For example, the predetermined condition may be that each of the at least one loss value is less than a predetermined loss threshold.

For example, in some embodiments, the calculating at least one loss value using a loss function corresponding to the neural network based on the mapping table, the at least one position parameter set, and the at least one output result in step S102 may include: for each position parameter set: determining a target result corresponding to the position parameter set based on the position parameter set; obtaining the N current position vectors in the mapping table; and calculating one loss value using the loss function corresponding to the neural network based on the N current position vectors, the target result, and an output result corresponding to a position vector set corresponding to the position parameter set.

For example, the plurality of current position vectors may be corrected based on the at least one loss value by using various optimization methods. For example, the optimization methods may include an adaptive moment estimation (Adam) optimization algorithm, a stochastic gradient descent (SGD) optimization algorithm, etc.

For example, in some embodiments, the plurality of current position vectors may be corrected once by using each loss value. In some other embodiments, the plurality of current position vectors may be corrected once by using the at least one loss value together. For example, the at least one loss value is averaged to obtain an average loss value, and then the plurality of current position vectors are corrected once based on the average loss value.

For example, in some embodiments, the loss function includes an error loss function and a smoothness loss function.

The position parameters such as the spacing between the atoms and the bond angle (an included angle between chemical bonds) are continuous, and the plurality of learnable position vectors obtained through learning should be smooth. Therefore, in some embodiments of the present disclosure, a smoothness regularization technique may be used; that is, the smoothness loss function is set. Setting the smoothness loss function is equivalent to providing specific regularization for the model (for example, the above neural network). Because the smoothness loss function restricts a relative change between the learnable position vectors, flexibility of the model is restricted. Such regularization can prevent overfitting of the model to some extent. In addition, the plurality of learnable position vectors obtained through learning are smoother, so that interpretability of the model is improved.

For example, in some embodiments, N is greater than 1, and the smoothness loss function is expressed as:

$${L}_{smooth} = \frac{{\sum }_{i = 1}^{N - 1} ‖ h ( {x}_{i + 1} ) - h ( {x}_{i} ) ‖}{{\sum }_{i = 1}^{N - 1} ‖ h ( {x}_{i} ) ‖} ,$$

where Lₛₘₒₒₜₕ represents the smoothness loss function, ∥*∥ represents a 2-norm, Σ represents summation, h(xᵢ₊₁) represents an (i+1)th current position vector in the N current position vectors, h(xᵢ) represents an ith current position vector in the N current position vectors, and i is a positive integer and is less than or equal to (N−1).

For example, in some embodiments, the error loss function includes a root mean square error loss function or a mean absolute error loss function.

For example, the root mean square error loss function is expressed as:

$${L}_{RMSE} = \sqrt{\frac{1}{M} {\sum }_{j = 1}^{M} {( {y}_{prej} - {y}_{labj} )}^{2}} ,$$

where LRMSE represents the root mean square error loss function, both M and j are positive integers, M represents the number of the at least one position parameter set, yprej represents an output result corresponding to a jth position parameter set in the at least one position parameter set, and ylabj represents a target result corresponding to the jth position parameter set in the at least one position parameter set.

For example, the mean absolute error loss function is expressed as:

$${L}_{MAE} = \frac{1}{M} {\sum }_{k = 1}^{M} \vert {y}_{prek} - {y}_{labk} \vert ,$$

where LMAE represents the mean absolute error loss function, both M and k are positive integers, M represents the number of the at least one position parameter set, yprek represents an output result corresponding to a kth position parameter set in the at least one position parameter set, and ylabk represents a target result corresponding to the kth position parameter set in the at least one position parameter set.

For example, the loss function is expressed as:

$$L = {L}_{e} + λ * {L}_{smooth} ,$$

where L represents the loss function, L_{c} represents the error loss function, Lₛₘₒₒₜₕ represents the smoothness loss function, and X represents a hyperparameter that controls a contribution of the smoothness loss function, and is a constant.

For example, Lₛₘₒₒₜₕ may be used as a regularization term. The smoothness loss function reduces position encoding flexibility, which is conducive to generalization of the model in a case of a small amount of data.

It should be noted that in some other embodiments, the loss function may include only the error loss function.

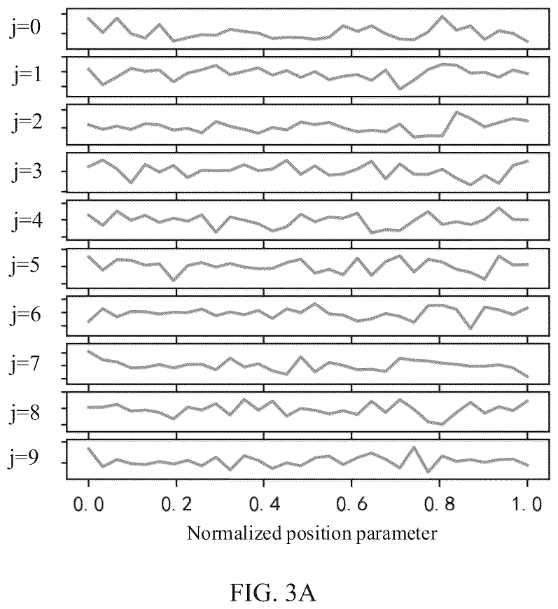

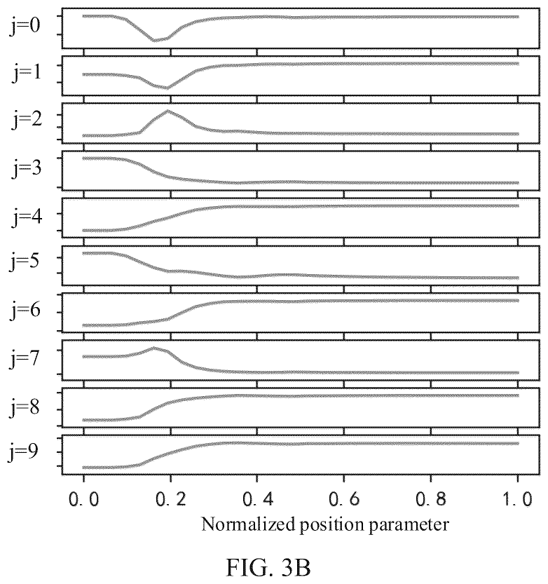

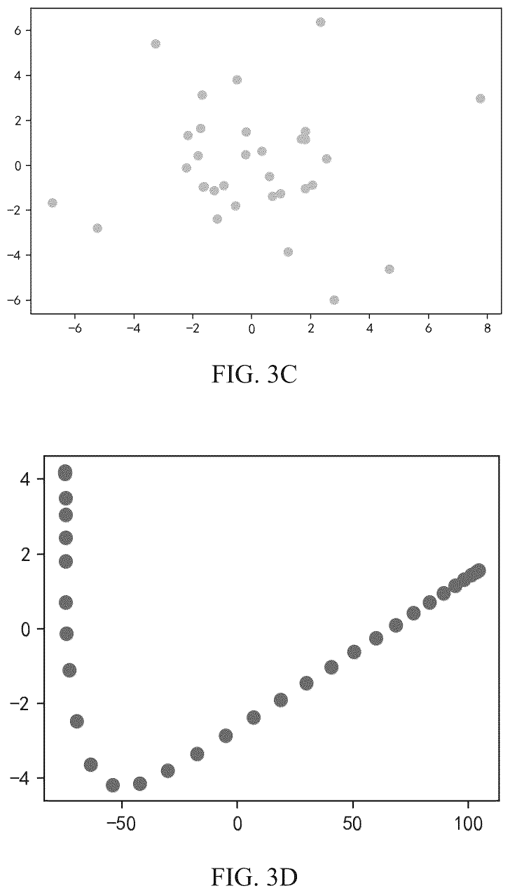

FIG. 3A is a schematic diagram of a curve formed by a plurality of learnable position vectors according to some embodiments of the present disclosure. FIG. 3B is a schematic diagram of a curve formed by a plurality of learnable position vectors according to some embodiments of the present disclosure. It should be noted that FIG. 3A and FIG. 3B show only ten dimensions, but each learnable position vector may alternatively include more dimensions. This is not limited in the present disclosure.

For example, a “normalized position parameter” in FIG. 3A and FIG. 3B may be a normalized distance.

As shown in FIG. 3A and FIG. 3B, a dimensionality of each learnable position vector is 10, and j represents a dimension, where j=0 represents a first dimension of the learnable position vector, and a curve corresponding to j=0 represents a curve formed by elements of the learnable position vectors in the first dimension; j=1 represents a second dimension of the learnable position vector, and a curve corresponding to j=1 represents a curve formed by elements of the learnable position vectors in the second dimension; and by analogy, j=9 represents a tenth dimension of the learnable position vector, and a curve corresponding to j=9 represents a curve formed by elements of the learnable position vectors in the tenth dimension.

For example, a curve chart in FIG. 3A represents learnable position vectors obtained without smoothness regularization; that is, the loss function includes only the error loss function. A curve chart in FIG. 3B represents learnable position vectors obtained through smoothness regularization; that is, the loss function includes the error loss function and the smoothness loss function. As shown in FIG. 3A, when the loss function includes only the error loss function, a value of the element of each learnable position vector in each dimension varies greatly with a distance, and a curve corresponding to each dimension is unsmooth. As shown in FIG. 3B, when the loss function includes the error loss function and the smoothness loss function, a value of the element of each learnable position vector in each dimension varies slightly with a distance, and a curve corresponding to each dimension is smooth.

It should be noted that in this embodiment of the present disclosure, the “smoothness regularization” represents addition of the smoothness loss function to the loss function.

Smoothness regularization can filter out an unwanted vector fluctuation of the learnable position vector, so that a feature of the learnable position vector obtained through learning is analyzed more easily. As shown in FIG. 3A and FIG. 3B, the following physical properties of the molecule can be observed based on the learnable position vector obtained through smoothness regularization. First, a derivative g of the learnable position vector with respect to the position parameter may reflect dependence of the corresponding molecular property on a scale of the position parameter (for example, a distance). For example, for molecular energy, g is close to zero when the position parameter is large. This indicates that in this dataset (the learnable position vectors shown in FIG. 3A and FIG. 3B are obtained through learning based on the dataset), the molecular energy is mainly determined by close interactions. Second, non-monotonicity of the learnable position vector is often correlated with non-monotonicity of the corresponding molecular property with respect to the position parameter. Third, if learnable position vectors in the mapping table respectively corresponding to a plurality of molecular properties are similar, the physical nature of the plurality of molecular properties is also similar.

For example, it can be learned based on FIG. 3A and FIG. 3B that if smoothness regularization is not performed, the obtained learnable position vector does not show an obvious property. Learnable position vectors corresponding to two position parameters differ greatly. If smoothness regularization is performed, the obtained learnable position vector is smoother and shows a simpler property. As shown in FIG. 3B, the learnable position vector is usually nonlinear and non-monotonic. For a learnable position vector generated for a specific property, it can be observed that turning points mainly occur at a small normalized position parameter (for example, about 0.2), and the learnable position vector changes little at a large normalized position parameter (for example, 0.4 to 1). For example, when the normalized position parameter ranges from 0.4 to 1, the learnable position vector corresponding to each position parameter hardly changes.

FIG. 3C is a schematic diagram of the plurality of learnable position vectors, shown in FIG. 3A, mapped to a two-dimensional space. FIG. 3D is a schematic diagram of the plurality of learnable position vectors, shown in FIG. 3B, mapped to a two-dimensional space. In FIG. 3C and FIG. 3D, horizontal axis and vertical axis coordinates respectively indicate two principal components obtained through principal component analysis (PCA) dimensionality reduction, where a horizontal axis indicates a first principal component obtained through PCA, and a vertical axis indicates a second principal component obtained through PCA.

FIG. 3C is a schematic diagram obtained by performing principal component analysis on the plurality of learnable position vectors shown in FIG. 3A and visualizing in the two-dimensional (2D) space. FIG. 3D is a schematic diagram obtained by performing principal component analysis on the plurality of learnable position vectors shown in FIG. 3B and visualizing in the 2D space. As shown in FIG. 3C, the learnable position vector obtained without smoothness regularization does not present a clear manifold, and the learnable position vector obtained through smoothness regularization forms a low-dimensional manifold. The low-dimensional manifold is not a straight line, but is consistent with non-linearity and non-monotonicity observed based on FIG. 3A and FIG. 3B.

For example, in some embodiments, step S11 may include: replacing values of the N current position vectors in the mapping table with values of the N learnable position vectors, respectively, to obtain the N updated current position vectors. In other words, values of the N updated current position vectors are respectively the values of the N learnable position vectors obtained through learning.

For example, for each current position vector in the mapping table, the value of the current position vector is updated with a value of a learnable position vector in the N learnable position vectors that corresponds to the current position vector, to obtain an updated current position vector.

For example, in some embodiments, the vector generation method further includes: determining a position parameter range based on a training dataset; and determining the N position parameters based on the position parameter range.

For example, the training dataset includes a plurality of pieces of training data.

For example, in some embodiments, the determining a position parameter range based on a training dataset includes: determining a plurality of training position parameters based on the plurality of pieces of training data; determining a first training position parameter and a second training position parameter in the plurality of training position parameters; and determining the position parameter range based on the first training position parameter and the second training position parameter.

For example, in the plurality of training position parameters, the first training position parameter is the largest, and the second training position parameter is the smallest. A minimum value of the position parameter range is the second training position parameter. A maximum value of the position parameter range is the first training position parameter.

For example, the N position parameters are evenly distributed in the position parameter range. For example, the N position parameters may be selected from the position parameter range at equal spacings. In other words, a difference between any two adjacent position parameters in the N position parameters is fixed.

For example, in some embodiments, an example in which the position parameter is the spacing between the two atoms is used. A plurality of training spacings are determined based on the plurality of pieces of training data. Then, a first training spacing and a second training spacing are selected from the plurality of training spacings. In the plurality of training spacings, a value of the first training spacing is the largest, and a value of the second training spacing is the smallest. Then, N segment nodes are evenly set between the first training spacing and the second training spacing. Each segment node correspondingly represents one spacing. For example, the N segment nodes include a segment node corresponding to the first training spacing and a segment node corresponding to the second training space; that is, the N spacings (that is, the N position parameters) include the first training spacing and the second training spacing.

It should be noted that the N position parameters may not be evenly distributed in the position parameter range. This may be specifically set based on the actual situation. For example, for a spacing r between atoms in a molecule, r is distributed in a spacing range [a, b], and in the spacing range [a, b], a spacing r between atoms in most molecules is distributed in a spacing range [c, b]. Therefore, more position parameters may be set in the spacing range [a, c], and fewer position parameters may be set in the spacing range [c, b], where a, b, and c are positive real numbers, c is less than b, and an absolute value of a difference between a and c may be less than that of a difference between c and b. For example, a may be 0, b may be 100, and c may be 10.

For example, in some embodiments, the determining a position parameter range based on a training dataset includes: determining a plurality of training position parameters based on the plurality of pieces of training data; determining a first training position parameter and a second training position parameter in the plurality of training position parameters; separately performing transformation on the first training position parameter and the second training position parameter to obtain a first transformed training position parameter and a second transformed training position parameter; and determining the position parameter range based on the first transformed training position parameter and the second transformed training position parameter.

For example, in the plurality of training position parameters, the first training position parameter is the largest, and the second training position parameter is the smallest. A minimum value of the position parameter range is the second transformed training position parameter. A maximum value of the position parameter range is the first transformed training position parameter.

For example, the transformation is performed on the N position parameters to obtain N transformed position parameters, and the N transformed position parameters are evenly distributed in the position parameter range.

For example, when the N position parameters are unevenly distributed, the transformation may be performed on the training position parameters to obtain the position parameter range. Then, values are selected from the position parameter range at equal spacings to obtain the N transformed position parameters. Finally, the N transformed position parameters are converted into the N position parameters. In this case, the N position parameters are unevenly distributed.

For example, in some embodiments, an example in which the position parameter is the spacing between the two atoms is used. A plurality of training spacings are determined based on the plurality of pieces of training data. Then, a first training spacing and a second training spacing are selected from the plurality of training spacings. In the plurality of training spacings, a value of the first training spacing is the largest, and a value of the second training spacing is the smallest. Then, the transformation is separately performed on the first training spacing and the second training spacing to obtain a first transformed training spacing and a second transformed training spacing. Finally, N segment nodes are set between the first transformed training spacing and the second transformed training spacing. Each segment node correspondingly represents one transformed training spacing. For example, reverse processing of the transformation is performed on the segment node to obtain a training spacing corresponding to the segment node. The N segment nodes include a segment node corresponding to the first transformed training spacing and a segment node corresponding to the second transformed training space; that is, the N transformed spacings include the first transformed training spacing and the second transformed training spacing.

For example, the transformation may be a logarithmic function. In this case, reverse processing of the logarithmic function is an exponential function. For example, a base of an exponential function may be the same as that of a logarithmic function. In some embodiments, the base of the logarithmic function may be 10. In this case, the transformation may be expressed as ln(1+r), where r is the first training position parameter or the second training position parameter. It should be noted that the base of the logarithmic function may be another value. This is not limited in the present disclosure.

It should be noted that in some embodiments, the minimum value of the position parameter range may be 0. In this case, only the first training position parameter (the first training position parameter is the largest) may be selected from the plurality of training position parameters. Then, the position parameter range may be determined based on the first training position parameter and 0. In this case, the minimum value of the position parameter range is 0, and the maximum value of the position parameter range is the first training position parameter.

For example, in some embodiments, when the learnable processing is performed, the neural network may be a trained neural network. However, the present disclosure is not limited thereto. In some other embodiments, the neural network may be a neural network to be trained. For example, in the vector generation method provided in this embodiment of the present disclosure, not only can the current position vector in the mapping table be updated, but also a parameter of the neural network can be corrected.

For example, in some embodiments, the vector generation method further includes: correcting the parameter of the neural network based on the at least one loss value.

For example, in the vector generation method provided in this embodiment of the present disclosure, the learnable processing may be performed multiple times, until the at least one loss value calculated based on the at least one position vector set randomly selected from the mapping table satisfies the predetermined condition. In this case, training of the neural network has also been completed. The trained neural network and the final mapping table correspond to each other. The trained neural network may be used to predict one property of the molecule; that is, the final mapping table corresponds to one property of the molecule.

For a plurality of properties of the molecule, a plurality of mapping tables respectively corresponding to the plurality of properties may be generated according to the vector generation method provided in this embodiment of the present disclosure. In addition, a plurality of neural networks respectively corresponding to the plurality of properties may be generated according to the vector generation method provided in this embodiment of the present disclosure. However, the present disclosure is not limited thereto. The plurality of neural networks may alternatively be generated by using another suitable method. This is not limited in this embodiment of the present disclosure. For example, structures of the plurality of neural networks corresponding to the plurality of properties of the molecule may be the same, but parameters of the plurality of neural networks are different. For another example, both structures and parameters of the plurality of neural networks corresponding to the plurality of properties of the molecule may be at least partially different.

For example, each time the learnable processing is performed, only some current position vectors in the mapping table are processed, or all the current position vectors in the mapping table may be processed.

It should be noted that when the final mapping table is generated, some current position vectors in the mapping table may not be selected for processing (training), and test costs of the untrained current position vectors are high. However, in the present disclosure, the addition of the smoothness loss function helps keep each current position vector smooth. Therefore, even though not directly trained, some current position vectors may be updated with changes of current position vectors adjacent to these current position vectors. In other words, all the current position vectors in the mapping table are updated during each update, so that some current position vectors can be updated even though not be selected. Therefore, test costs of all current position vectors in the final mapping table are low, and a user requirement is met.

For example, each time the learnable processing is performed, the at least one piece of data to be processed may be selected from the training dataset randomly or according to a specific rule, to determine the at least one position parameter set. It can be learned from this that for example, a position parameter set determined when the learnable processing is performed for the first time may be at least partially different from that determined when the learnable processing is performed for the second time, or a position parameter set determined when the learnable processing is performed for the first time may be the same as that determined when the learnable processing is performed for the second time.

For example, in some embodiments, when the learnable processing is performed for the first time, the learnable processing may include: determining A1 position parameter sets; obtaining A1 position vector sets respectively corresponding to the A1 position parameter sets based on the mapping table (for example, described as a first mapping table below); and processing the A1 position vector sets to obtain N learnable position vectors. The processing the A1 position vector sets to obtain N learnable position vectors includes: separately processing the A1 position vector sets using a neural network (for example, described as a first neural network below) to obtain A1 output results respectively corresponding to the A1 position vector sets; calculating A1 loss values using a loss function corresponding to the first neural network based on the first mapping table, the A1 position parameter sets, and the A1 output results; and in response to the A1 loss values not satisfying the predetermined condition, correcting N current position vectors in the first mapping table based on the A1 loss values to obtain the N learnable position vectors; or in response to the A1 loss values satisfying the predetermined condition, using the N current position vectors in the first mapping table as the N learnable position vectors. Then, the first mapping table is updated with the N learnable position vectors obtained when the learnable processing is performed for the first time, to obtain a mapping table (for example, described as a second mapping table below) updated for the first time. In addition, a parameter of the first neural network is further corrected based on the A1 loss values to obtain a neural network (for example, described as a second neural network below) corrected for the first time.

For example, when the learnable processing is performed for the second time, the learnable processing may include: determining A2 position parameter sets; obtaining A2 position vector sets respectively corresponding to the A2 position parameter sets based on the second mapping table; and encoding the A2 position vector sets to obtain N learnable position vectors. The encoding the A2 position vector sets to obtain N learnable position vectors includes: separately processing the A2 position vector sets using the second neural network to obtain A2 output results respectively corresponding to the A2 position vector sets; calculating A2 loss values using a loss function corresponding to the second neural network based on the second mapping table, the A2 position parameter sets, and the A2 output results; and in response to the A2 loss values not satisfying the predetermined condition, correcting N current position vectors in the second mapping table based on the A2 loss values to obtain the N learnable position vectors; or in response to the A2 loss values satisfying the predetermined condition, using the N current position vectors in the second mapping table as the N learnable position vectors. Then, the second mapping table is updated with the N learnable position vectors obtained when the learnable processing is performed for the second time, to obtain a mapping table updated for the second time. In addition, a parameter of the second neural network is further corrected based on the A2 loss values to obtain a neural network corrected for the second time. By analogy, the learnable processing may be performed for the third time, the fourth time, and the like. Each time the learnable processing is performed, the mapping table is an updated mapping table obtained after the learnable processing is performed last time, and the neural network is a corrected neural network obtained after the learnable processing is performed last time, until the final mapping table and a final neural network satisfy the following condition: After at least one position vector set randomly selected from the final mapping table is processed using the final neural network to obtain at least one output result, at least one loss value calculated using a loss function corresponding to the final neural network based on the final mapping table, the at least one position parameter set, and the at least one output result satisfies the predetermined condition.

For example, a smoothness loss function in a loss function corresponding to the first neural network performs calculation using each current position vector in the first mapping table, and a smoothness loss function in a loss function corresponding to the second neural network performs calculation using each current position vector in the second mapping table.

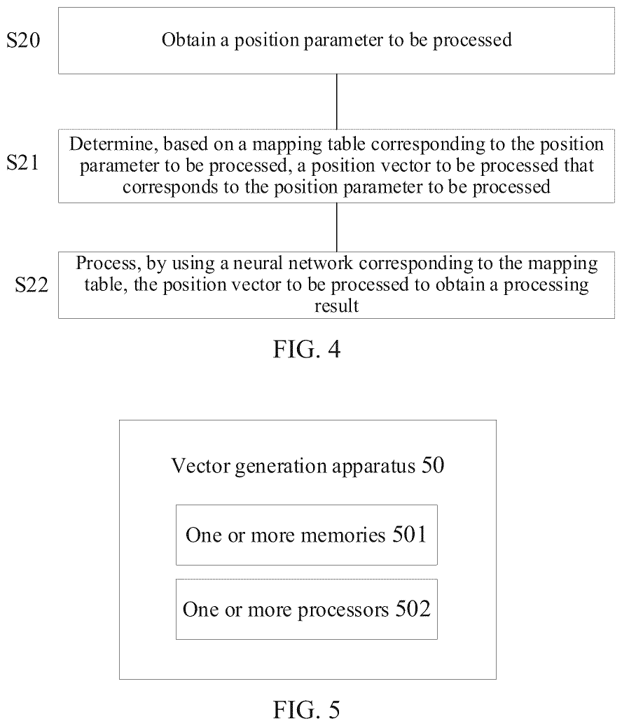

At least one embodiment of the present disclosure further provides a data processing method. FIG. 4 is a schematic flowchart of a data processing method according to at least one embodiment of the present disclosure.

As shown in FIG. 4, the data processing method may include the following steps S20 to S22.

In step S20, a position parameter to be processed is obtained.

In step S21, a position vector to be processed that corresponds to the position parameter to be processed is determined based on a mapping table corresponding to the position parameter to be processed.

For example, the mapping table includes N current position vectors, and the N current position vectors in the mapping table are obtained through updating based on the N learnable position vectors obtained according to the vector generation method described in any one of the embodiments of the present disclosure. For example, the mapping table may be a final mapping table obtained by using the vector generation method described in any one of the embodiments of the present disclosure; that is, the mapping table in step S21 satisfies the following condition: After at least one position vector set randomly selected from the mapping table is processed using a neural network corresponding to the mapping table to obtain at least one output result, at least one loss value calculated using a loss function corresponding to the neural network corresponding to the mapping table based on the mapping table, at least one position parameter set, and the at least one output result satisfies a predetermined condition.

For example, the mapping table further includes N position parameters in a one-to-one correspondence with the N current position vectors.

In step S22, the position vector to be processed is processed by using the neural network corresponding to the mapping table to obtain a processing result.

The data processing method provided in this embodiment of the present disclosure may be applied to molecular property prediction. The processing result (that is, a property of a molecule) obtained by using the data processing method is more accurate. For example, in the field of new drug research and development, predicting the property of the molecule more accurately can find better candidate drugs faster with lower costs and greatly increase a drug research and development speed.

For example, both the mapping table and the neural network may be determined based on a property of the molecule to be predicted. For example, when a dipole moment of the molecule needs to be predicted, a mapping table and a neural network that correspond to the dipole moment may be selected. When an energy gap (obtained by subtracting E(highest occupied molecule orbital (HOMO)) from E(lowest unoccupied molecule orbital (LUMO))) of the molecule needs to be predicted, a mapping table and a neural network that correspond to the energy gap may be selected. The mapping table corresponding to the dipole moment is different from that corresponding to the energy gap. A structure of the neural network corresponding to the dipole moment may be the same as that of the neural network corresponding to the energy gap, but parameters of the neural network corresponding to the dipole moment are at least partially different from those of the neural network corresponding to the energy gap.

For example, the position parameter to be processed includes a spacing between two adjacent atoms in the molecule and/or a bond angle in the molecule. The processing result may include a predicted value of the property of the molecule to be predicted.

It should be noted that if the position parameter to be processed is greater than a largest position parameter in the mapping table, the position parameter to be processed may be considered as being the same as the largest position parameter in the mapping table. Similarly, if the position parameter to be processed is less than a smallest position parameter in the mapping table, the position parameter to be processed may be considered as being the same as the smallest position parameter in the mapping table.

For example, a number of position parameters in the mapping table is finite, while the spacing or the bond angle is an infinite and continuous value. When the position vector to be processed is determined, if the position parameter to be processed is the same as a position parameter in the position parameters in the mapping table, a current position vector corresponding to the position parameter in the mapping table may be directly used as the position vector to be processed that corresponds to the position parameter to be processed; or if the position parameter to be processed is different from any position parameter in the mapping table, the position vector to be processed that corresponds to the position parameter to be processed may be determined through interpolation, such that position vectors are continuous for the position parameters.

For example, in some embodiments, in step S21, when the position parameter to be processed is different from any position parameter in the N position parameters in the mapping table corresponding to the position parameter to be processed, two position parameters to be interpolated that correspond to the position parameter to be processed are determined. The two position parameters to be interpolated are two of the N position parameters, and the position parameter to be processed is located between the two position parameters to be interpolated. Then, two current position vectors corresponding to the two position parameters to be interpolated are determined, based on the mapping table, as two position vectors to be interpolated. Finally, interpolation is performed on the two position vectors to be interpolated to obtain the position vector to be processed that corresponds to the position parameter to be processed. When the position parameter to be processed is one of the N position parameters in the mapping table corresponding to the position parameter to be processed, a current position vector corresponding to the position parameter to be processed is directly determined, based on the mapping table, as the position vector to be processed.

For example, an interpolation method corresponding to the interpolation includes linear interpolation and/or cubic Hermite interpolation.

Table 2 shows test results obtained by testing different properties of the molecule on two datasets.

For example, as shown in Table 2, the two datasets are the QM9 dataset (Ramakrishna et al., 2014) and the PubChemQC PM6 dataset (Nakata et al., 2020).

TABLE 2

| Dataset | QM9 | | | | | | | | | | | | PM6 |
|---|---|---|---|---|---|---|---|---|---|---|---|---|---|
| Task | α | Δ∈ | ∈_{HOMO} | ∈_{LUMO} | μ | Cᵥ | G | H | R² | U | U₀ | ZPVE | etot |
| Unit | bohr³ | meV | meV | meV | D | cal/mol K | meV | meV | bohr³ | meV | meV | meV | meV |
| NMP | .092 | 69 | 43 | 38 | .030 | .040 | 19 | 17 | .180 | 20 | 20 | 1.50 | — |
| SchNet | .235 | 63 | 41 | 34 | .033 | .033 | 14 | 14 | .073 | 19 | 14 | 1.70 | — |
| Cormorant | .085 | 61 | 34 | 38 | .038 | .026 | 20 | 21 | .961 | 21 | 22 | 2.03 | — |
| L1Net. | .088 | 68 | 46 | 35 | .043 | .031 | 14 | 14 | .354 | 14 | 13 | 1.56 | — |
| LieConv. | .084 | 49 | 30 | 25 | .032 | .038 | 22 | 24 | .800 | 19 | 19 | 2.28 | — |
| DimeNet++ | .048 | 44 | 27 | 21 | .032 | .023 | 7 | 7 | .334 | 7 | 7 | 1.18 | 9 |
| +PosEnc | .048 | 44 | 26 | 20 | .030 | .023 | 7 | 7 | .260 | 7 | 7 | 1.18 | 9 |
| +Smooth | .048 | 44 | 24 | 20 | .028 | .023 | 7 | 7 | .260 | 7 | 7 | 1.18 | 9 |
| EGNN | .071 | 48 | 29 | 25 | .029 | .031 | 12 | 12 | .106 | 12 | 11 | 1.55 | 11 |
| +PosEnc | .063 | 44 | 26 | 22 | .027 | .028 | 10 | 10 | .086 | 10 | 9 | 1.49 | 9 |
| +Smooth | .062 | 43 | 27 | 23 | .023 | .028 | 9 | 9 | .091 | 9 | 9 | 1.50 | 9 |

For example, the QM9 dataset has been a benchmark for a molecular chemical property prediction task. The QM9 dataset includes DFT calculation results of 134,000 stable organic small molecules. These molecules include five elements (CHONF), and each element contains an average of 18 atoms. A calculation result of the PM6 dataset is obtained by using a semi-empirical method. A number of molecules in the PM6 dataset is about 221 million. In this test, molecule samples in the PM6 dataset are filtered, such that the molecules in the PM6 dataset include the five elements included in the molecule in the QM9 dataset, and each molecule contains less than 100 atoms.

For example, as shown in Table 2, in this test, a total of 13 properties (that is, task units) of the molecule are tested. The 13 properties are isotropic polarizability (a), HOMO energy (εHOMO), LUMO energy (εLUMO), energy gap Δε (Δε=εHOMO−εLUMO), dipole moment (μ), thermal capacity (Cv), free energy (G), enthalpy change (H), electron space range (R2), internal energy at 298.15K (U), internal energy at 0K (U0), zero-point vibration energy (ZPVE), and total energy (etot).

In Table 2, the mapping table obtained in this embodiment of the present disclosure is used in two existing models: DimeNet++(Dime stands for dual independent map model) and exploiting edge features for graph neural networks (EGNN). “+PosEnc” means that a plurality of current position vectors (vector functions corresponding to the plurality of current position vectors are continuous and derivable), generated by using the vector generation method provided in the embodiments of the present disclosure, in the mapping table are used as an input to the existing model (DimeNet++ or EGNN). “+Smooth” means that a “smoothness regularization” technique provided in the embodiments of the present disclosure is used, in addition to “+PosEnc”; that is, a smoothness loss function is added in a process of generating the plurality of current position vectors in the mapping table. It should be noted that properties separately corresponding to DimeNet++ and EGNN indicate test results obtained without using the plurality of current position vectors, generated in the present disclosure, in the mapping table as the input.

Each number in Table 2 indicates an error of each model on a test dataset, and a smaller number indicates higher model accuracy. With the technical solution provided in this embodiment of the present disclosure, accuracy of both DimeNet++ and EGNN is improved, and a test error is reduced.

It can be learned from Table 2 that compared with the existing models (for example, the NMP (Gilmer et al., 2017) model, the SchNet (Schuutt et al., 2017) model, the Cormorant (Anderson et al., 2019) model, the L1Net (Miller et al., 2020) model, the LieConv (Finzi et al., 2020) model, the DimeNet++model, and the EGNN model), when a machine learning model uses the current position vector in the mapping table obtained in this embodiment of the present disclosure as an input, an error of the machine learning model on the test dataset can be reduced, and accuracy of an output result of the machine learning model can be improved.

It should be noted that each machine learning model has the same hyperparameter in this test.

FIG. 5 is a schematic diagram of a vector generation apparatus according to at least one embodiment of the present disclosure.

As shown in FIG. 5, some embodiments of the present disclosure further provide a vector generation apparatus 50. The vector generation apparatus 50 includes one or more memories 501 and one or more processors 502. It should be noted that the components of the vector generation apparatus 50 shown in FIG. 5 are merely exemplary and non-restrictive. Based on an actual application requirement, the vector generation apparatus 50 may further include other components.

For example, the one or more memories 501 are configured to store computer-executable instructions in a non-transitory manner. The one or more processors 502 are configured to run the computer-executable instructions. When the computer-executable instructions are run by the one or more processors 502, the vector generation method according to any one of the embodiments of the present disclosure is implemented. For specific implementation and related explanation content of each step of the vector generation method, reference may be made to the embodiment of the vector generation method, and details of the same parts are not described herein.

For example, the processor 502 and the memory 501 may directly or indirectly communicate with each other.

For example, the processor 502 and the memory 501 may communicate through a network. The network may include a wireless network, a wired network, and/or any combination of a wireless network and a wired network. The processor 502 and the memory 501 may also communicate with each other through a system bus. This is not limited in the present disclosure.

For example, the processor 502 and the memory 501 may be disposed at a server (or a cloud).

For example, the processor 502 may control another component in the vector generation apparatus 50 to perform a desired function. The processor 502 may be a central processing unit (CPU), a graphics processing unit (GPU), a network processor (NP), or the like. Alternatively, the processor 502 may be another form of processing unit with a data processing capability and/or a program execution capability, for example, a digital signal processor (DSP), an application-specific integrated circuit (ASIC), a field programmable gate array (FPGA), a tensor processing unit (TPU) or other programmable logic devices, a discrete gate or transistor logic device, or a discrete hardware component. The central processing unit (CPU) may have an X86 or ARM architecture or the like.

For example, the memory 501 may include any combination of one or more computer program products, which may include various forms of computer-readable storage media, such as a volatile memory and/or a non-volatile memory. The volatile memory may include, for example, a random access memory (RAM) and/or a cache memory (cache). The non-volatile memory may include, for example, a read-only memory (ROM), a hard disk, an erasable programmable read-only memory (EPROM), a portable compact disk read-only memory (CD-ROM), a USB memory, and a flash memory. One or more computer-readable instructions may be stored in the computer-readable storage medium. The processor 502 may execute the computer-readable instructions to implement various functions of the vector generation apparatus 50. Various applications, various data, and the like may further be stored in the storage medium.

For technical effects achievable by the vector generation apparatus, reference may be made to the related description in the embodiment of the vector generation method, and details of the same parts are not described herein.

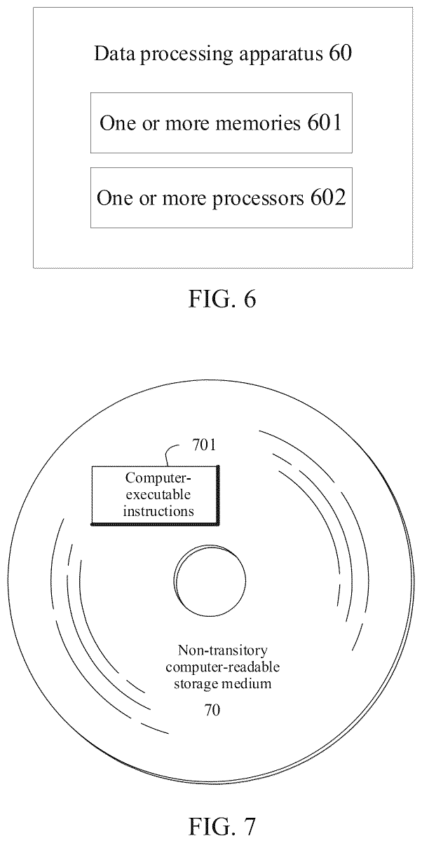

FIG. 6 is a schematic diagram of a data processing apparatus according to at least one embodiment of the present disclosure.

As shown in FIG. 6, some embodiments of the present disclosure further provide a data processing apparatus 60. The data processing apparatus 60 includes one or more memories 601 and one or more processors 602. It should be noted that the components of the data processing apparatus 60 shown in FIG. 6 are merely exemplary and non-restrictive. Based on an actual application requirement, the data processing apparatus 60 may further include other components.

For example, the one or more memories 601 are configured to store computer-executable instructions in a non-transitory manner. The one or more processors 602 are configured to run the computer-executable instructions. When the computer-executable instructions are run by the one or more processors 602, the data processing method according to any one of the embodiments of the present disclosure is implemented. For specific implementation and related explanation content of each step of the data processing method, reference may be made to the embodiment of the data processing method, and details of the same parts are not described herein.

For example, the processor 602 and the memory 601 may directly or indirectly communicate with each other.

For example, the processor 602 and the memory 601 may communicate through a network. The network may include a wireless network, a wired network, and/or any combination of a wireless network and a wired network. The processor 602 and the memory 601 may also communicate with each other through a system bus. This is not limited in the present disclosure.

For example, the processor 602 and the memory 601 may be disposed at a server (or a cloud).

For example, the processor 602 may control another component in the data processing apparatus 60 to perform a desired function. The processor 602 may be a central processing unit (CPU), a graphics processing unit (GPU), a network processor (NP), or the like. Alternatively, the processor 602 may be another form of processing unit with a data processing capability and/or a program execution capability, for example, a digital signal processor (DSP), an application-specific integrated circuit (ASIC), a field programmable gate array (FPGA), a tensor processing unit (TPU) or other programmable logic devices, a discrete gate or transistor logic device, or a discrete hardware component. The central processing unit (CPU) may have an X86 or ARM architecture or the like.

For example, the memory 601 may include any combination of one or more computer program products, which may include various forms of computer-readable storage media, such as a volatile memory and/or a non-volatile memory. The volatile memory may include, for example, a random access memory (RAM) and/or a cache memory (cache). The non-volatile memory may include, for example, a read-only memory (ROM), a hard disk, an erasable programmable read-only memory (EPROM), a portable compact disk read-only memory (CD-ROM), a USB memory, and a flash memory. One or more computer-readable instructions may be stored in the computer-readable storage medium. The processor 602 may execute the computer-readable instructions to implement various functions of the data processing apparatus 60. Various applications, various data, and the like may further be stored in the storage medium.

For technical effects achievable by the data processing apparatus, reference may be made to the related description in the embodiment of the data processing method, and details of the same parts are not described herein.

FIG. 7 is a schematic diagram of a non-transitory computer-readable storage medium according to at least one embodiment of the present disclosure. For example, as shown in FIG. 7, one or more computer-executable instructions 701 may be stored in a non-transitory computer-readable storage medium 70 in a non-transitory manner. For example, when the computer-executable instructions 701 are executed by a processor, one or more steps of the vector generation method according to any one of the embodiments of the present disclosure or one or more steps of the data processing method according to any one of the embodiments of the present disclosure may be performed.

For example, the non-transitory computer-readable storage medium 70 may be used in the vector generation apparatus 50 or the data processing apparatus 60. For example, the non-transitory computer-readable storage medium 70 may include the memory 501 of the vector generation apparatus 50 or the memory 601 of the data processing apparatus 60.

For example, for the description of the non-transitory computer-readable storage medium 70, reference may be made to the description of the memory 501 in the embodiment of the vector generation apparatus 50 or the description of the memory 601 in the embodiment of the data processing apparatus 60, and details of the same parts are not described again.

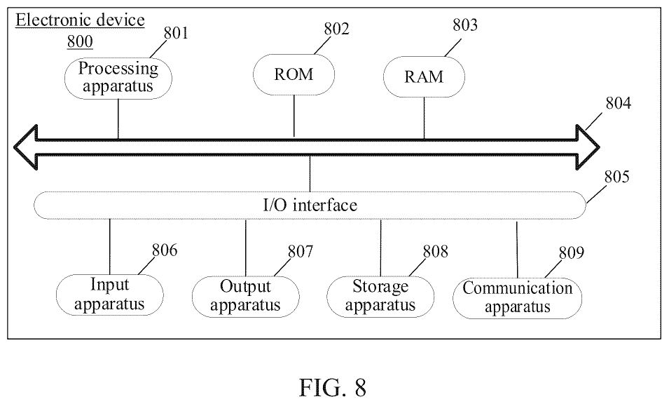

Reference is made to FIG. 8 below, which is a schematic diagram of a structure of an electronic device 800 suitable for implementing the embodiments of the present disclosure. The electronic device 800 may be a terminal device or a server, and may be configured to perform the vector generation method and/or the data processing method in the above embodiments. The electronic device in this embodiment of the present disclosure may include, but is not limited to, a mobile terminal such as a mobile phone, a notebook computer, a digital broadcast receiver, a personal digital assistant (PDA for short), a tablet computer (Portable Android Device, PAD for short), a portable media player (PMP for short), and a vehicle-mounted terminal (such as a vehicle navigation terminal), and a fixed terminal such as a television (TV), a desktop computer, and a smart home device. The electronic device shown in FIG. 8 is merely an example and should not impose any limitation on the function and scope of use of the embodiments of the present disclosure.

As shown in FIG. 8, the electronic device 800 may include a processing apparatus (e.g., a central processing unit, a graphics processing unit, etc.) 601 that may perform a variety of appropriate actions and processing in accordance with a program stored in a read-only memory (ROM) 802 or a program loaded from a storage apparatus 806 into a random access memory (RAM) 803. The RAM 803 further stores various programs and data required for the operation of the electronic device 800. The processing apparatus 801, the ROM 802, and the RAM 803 are connected to each other through a bus 804. An input/output (I/O) interface 805 is also connected to the bus 804.

Generally, the following apparatuses may be connected to the I/O interface 805: an input apparatus 806 including, for example, a touchscreen, a touchpad, a keyboard, a mouse, a camera, a microphone, an accelerometer, and a gyroscope; an output apparatus 807 including, for example, a liquid crystal display (LCD), a speaker, and a vibrator; the storage apparatus 806 including, for example, a tape and a hard disk; and a communication apparatus 809. The communication apparatus 809 may allow the electronic device 800 to perform wireless or wired communication with other devices to exchange data. Although FIG. 8 shows the electronic device 800 having various apparatuses, it should be understood that it is not required to implement or have all of the shown apparatuses. It may be an alternative to implement or have more or fewer apparatuses.

In particular, according to an embodiment of the present disclosure, the process described above with reference to the flowcharts may be implemented as a computer software program. For example, this embodiment of the present disclosure includes a computer program product, which includes a computer program carried on a non-transitory computer-readable medium, where the computer program includes program code for performing the method shown in the flowchart, to perform one or more steps of the data processing method described above. In such an embodiment, the computer program may be downloaded from a network through the communication apparatus 809 and installed, installed from the storage apparatus 806, or installed from the ROM 802. When the computer program is executed by the processing apparatus 801, the above-mentioned functions defined in the method of the embodiment of the present disclosure are performed.

It should be noted that in the context of the present disclosure, a computer-readable medium may be a tangible medium that may contain or store a program used by or in combination with an instruction execution system, apparatus, or device. The computer-readable medium may be a computer-readable signal medium, a computer-readable storage medium, or any combination thereof. Examples of the computer-readable storage medium may include, but are not limited to: electric, magnetic, optical, electromagnetic, infrared, or semiconductor systems, apparatuses, or devices, or any combination thereof. A more specific example of the computer-readable storage medium may include, but is not limited to: an electrical connection having one or more wires, a portable computer magnetic disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof. In the present disclosure, the computer-readable storage medium may be any tangible medium containing or storing a program which may be used by or in combination with an instruction execution system, apparatus, or device. In the present disclosure, the computer-readable signal medium may include a data signal propagated in a baseband or as a part of a carrier, the data signal carrying computer-readable program code. The propagated data signal may be in various forms, including but not limited to an electromagnetic signal, an optical signal, or any suitable combination thereof. The computer-readable signal medium may also be any computer-readable medium other than the computer-readable storage medium. The computer-readable signal medium may send, propagate, or transmit a program used by or in combination with an instruction execution system, apparatus, or device. The program code contained in the computer-readable medium may be transmitted by any suitable medium, including but not limited to: electric wires, optical cables, radio frequency (RF), and the like, or any suitable combination thereof.

The computer-readable medium may be contained in the electronic device. Alternatively, the computer-readable medium may exist independently, without being assembled into the electronic device.

The computer program code for performing the operations in the present disclosure may be written in one or more programming languages or a combination thereof, where the programming languages include, but are not limited to, an object-oriented programming language, such as Java, Smalltalk, and C++, and further include conventional procedural programming languages, such as “C” language or similar programming languages. The program code may be completely executed on a computer of a user, partially executed on a computer of a user, executed as an independent software package, partially executed on a computer of a user and partially executed on a remote computer, or completely executed on a remote computer or server. In the circumstance involving the remote computer, the remote computer may be connected to a computer of a user over any type of network, including a local area network (LAN) or a wide area network (WAN), or may be connected to an external computer (for example, connected over the Internet using an Internet service provider).

The flowcharts and block diagrams in the accompanying drawings illustrate the possibly implemented architecture, functions, and operations of the system, method, and computer program product according to various embodiments of the present disclosure. In this regard, each block in the flowchart or block diagram may represent a module, program segment, or part of code, and the module, program segment, or part of code contains one or more executable instructions for implementing the specified logical functions. It should also be noted that in some alternative implementations, the functions marked in the blocks may also occur in an order different from that marked in the accompanying drawings. For example, two blocks shown in succession may actually be executed substantially in parallel, or may sometimes be executed in a reverse order, depending on a function involved. It should also be noted that each block in the block diagram and/or the flowchart, and a combination of the blocks in the block diagram and/or the flowchart may be implemented by a dedicated hardware-based system that executes specified functions or operations, or may be implemented by a combination of dedicated hardware and computer instructions.

The related units described in the embodiments of the present disclosure may be implemented by software, or may be implemented by hardware. The name of a unit does not constitute a limitation on the unit in some cases.

The functions described herein above may be performed at least partially by one or more hardware logic components. For example, without limitation, exemplary types of hardware logic components that may be used include: a field programmable gate array (FPGA), an application-specific integrated circuit (ASIC), an application-specific standard product (ASSP), a system on a chip (SOC), a complex programmable logic device (CPLD), and the like.

In a first aspect, according to one or more embodiments of the present disclosure, there is provided a vector generation method, including: performing learnable processing on N current position vectors in a mapping table to obtain N learnable position vectors, where N is a positive integer; and updating the N current position vectors with the N learnable position vectors.

According to one or more embodiments of the present disclosure, the vector generation method further includes: performing the learnable processing on N updated current position vectors again.

According to one or more embodiments of the present disclosure, the updating the N current position vectors with the N learnable position vectors includes: replacing values of the N current position vectors in the mapping table with values of the N learnable position vectors, respectively.

According to one or more embodiments of the present disclosure, the mapping table indicates a mapping relationship between a position parameter and a current position vector, and further includes N position parameters in a one-to-one correspondence with the N current position vectors; and the learnable processing includes: determining at least one position parameter set, where each position parameter set includes at least one of the N position parameters; obtaining at least one position vector set respectively corresponding to the at least one position parameter set based on the mapping table, where each position vector set includes a current position vector that is obtained based on the mapping table and that corresponds to a position parameter in a position parameter set corresponding to the position vector set; and processing the at least one position vector set to obtain the N learnable position vectors.

According to one or more embodiments of the present disclosure, the processing the at least one position vector set to obtain the N learnable position vectors includes: separately processing the at least one position vector set using a neural network to obtain at least one output result respectively corresponding to the at least one position vector set; calculating at least one loss value using a loss function corresponding to the neural network based on the mapping table, the at least one position parameter set, and the at least one output result; and in response to the at least one loss value not satisfying a predetermined condition, correcting the N current position vectors based on the at least one loss value to obtain the N learnable position vectors; or in response to the at least one loss value satisfying the predetermined condition, using the N current position vectors as the N learnable position vectors.

According to one or more embodiments of the present disclosure, the calculating at least one loss value using a loss function corresponding to the neural network based on the mapping table, the at least one position parameter set, and the at least one output result includes: for each position parameter set: determining a target result corresponding to the position parameter set based on the position parameter set; obtaining the N current position vectors in the mapping table; and calculating one loss value using the loss function corresponding to the neural network based on the N current position vectors, the target result, and an output result corresponding to a position vector set corresponding to the position parameter set.

According to one or more embodiments of the present disclosure, the vector generation method further includes: correcting a parameter of the neural network based on the at least one loss value.

According to one or more embodiments of the present disclosure, the loss function includes an error loss function and a smoothness loss function.

According to one or more embodiments of the present disclosure, N is greater than 1, and the smoothness loss function is expressed as:

$${L}_{smooth} = \frac{{\sum }_{i = 1}^{N - 1} ‖ h ( {x}_{i + 1} ) - h ( {x}_{i} ) ‖}{{\sum }_{i = 1}^{N - 1} ‖ h ( {x}_{i} ) ‖} ,$$

where Lₛₘₒₒₜₕ represents the smoothness loss function, ∥*∥ represents a 2-norm, Σ represents summation, h(xᵢ₊₁) represents an (i+1)th current position vector in the N current position vectors, h(xᵢ) represents an ith current position vector in the N current position vectors, and i is a positive integer and is less than or equal to (N−1).

According to one or more embodiments of the present disclosure, the error loss function includes a root mean square error loss function or a mean absolute error loss function.

According to one or more embodiments of the present disclosure, the loss function is expressed as:

$$L = {L}_{e} + λ * {L}_{smooth} ,$$

where L represents the loss function, Lₑ represents the error loss function, Lₛₘₒₒₜₕ represents the smoothness loss function, and χ represents a hyperparameter for controlling a contribution of the smoothness loss function, and is a constant.

According to one or more embodiments of the present disclosure, the obtaining at least one position vector set respectively corresponding to the at least one position parameter set based on the mapping table includes: for each position parameter in each position parameter set: in response to the position parameter being different from any one of the N position parameters: determining a first position parameter to be interpolated and a second position parameter to be interpolated that correspond to the position parameter, where the first position parameter to be interpolated and the second position parameter to be interpolated are two of the N position parameters, and the position parameter is located between the first position parameter to be interpolated and the second position parameter to be interpolated; determining, based on the mapping table, a current position vector corresponding to the first position parameter to be interpolated as a first position vector to be interpolated and a current position vector corresponding to the second position parameter to be interpolated as a second position vector to be interpolated; and performing interpolation on the first position vector to be interpolated and the second position vector to be interpolated to obtain a current position vector corresponding to the position parameter; or in response to the position parameter being one of the N position parameters: determining a current position vector corresponding to the position parameter directly based on the mapping table.

According to one or more embodiments of the present disclosure, an interpolation method corresponding to the interpolation includes linear interpolation and/or cubic Hermite interpolation.

According to one or more embodiments of the present disclosure, the determining at least one position parameter set includes: determining at least one piece of data to be processed; and determining, based on the at least one piece of data to be processed, the at least one position parameter set in a one-to-one correspondence with the at least one piece of data to be processed.

According to one or more embodiments of the present disclosure, each piece of data to be processed corresponds to one molecule.

According to one or more embodiments of the present disclosure, the vector generation method further includes: determining a position parameter range based on a training dataset, where the training dataset includes a plurality of pieces of training data; and determining the N position parameters based on the position parameter range.

According to one or more embodiments of the present disclosure, the determining a position parameter range based on a training dataset includes: determining a plurality of training position parameters based on the plurality of pieces of training data; determining a first training position parameter and a second training position parameter in the plurality of training position parameters, where in the plurality of training position parameters, the first training position parameter is the largest, and the second training position parameter is the smallest; and determining the position parameter range based on the first training position parameter and the second training position parameter.

According to one or more embodiments of the present disclosure, the N position parameters are evenly distributed in the position parameter range.

According to one or more embodiments of the present disclosure, the determining a position parameter range based on a training dataset includes: determining a plurality of training position parameters based on the plurality of pieces of training data; determining a first training position parameter and a second training position parameter in the plurality of training position parameters, where in the plurality of training position parameters, the first training position parameter is the largest, and the second training position parameter is the smallest; separately performing transformation on the first training position parameter and the second training position parameter to obtain a first transformed training position parameter and a second transformed training position parameter; and determining the position parameter range based on the first transformed training position parameter and the second transformed training position parameter.

According to one or more embodiments of the present disclosure, the transformation is performed on the N position parameters to obtain N transformed position parameters, and the N transformed position parameters are evenly distributed in the position parameter range.

According to one or more embodiments of the present disclosure, each position parameter in the mapping table includes a spacing between two adjacent atoms in a molecule and/or a bond angle in the molecule.

In a second aspect, according to one or more embodiments of the present disclosure, there is provided a data processing method, including: obtaining a position parameter to be processed; determining, based on a mapping table corresponding to the position parameter to be processed, a position vector to be processed that corresponds to the position parameter to be processed, where the mapping table includes N current position vectors, and the N current position vectors in the mapping table are obtained through updating based on the N learnable position vectors obtained according to the vector generation method described in any one of the embodiments of the present disclosure; and processing, by using a neural network corresponding to the mapping table, the position vector to be processed to obtain a processing result.

According to one or more embodiments of the present disclosure, the position parameter to be processed includes a spacing between two adjacent atoms in a molecule and/or a bond angle in the molecule; and the processing result includes a predicted value of a property of the molecule.

In a third aspect, according to one or more embodiments of the present disclosure, there is provided a vector generation apparatus, including: one or more memories storing computer-executable instructions in a non-transitory manner; and one or more processors configured to run the computer-executable instructions, where when the computer-executable instructions are run by the one or more processors, the vector generation method according to any one of the embodiments of the present disclosure is implemented.

In a fourth aspect, according to one or more embodiments of the present disclosure, there is provided a data processing apparatus, including: one or more memories storing computer-executable instructions in a non-transitory manner; and one or more processors configured to run the computer-executable instructions, where when the computer-executable instructions are run by the one or more processors, the data processing method according to any one of the embodiments of the present disclosure is implemented.

In a fifth aspect, according to one or more embodiments of the present disclosure, there is provided a non-transitory computer-readable storage medium, where the non-transitory computer-readable storage medium stores computer-executable instructions, and when the computer-executable instructions are executed by a processor, the vector generation method according to any one of the embodiments of the present disclosure is implemented, or the data processing method according to any one of the embodiments of the present disclosure is implemented.

The above description illustrates merely preferred embodiments of the present disclosure and explanations of the applied technical principles. Those skilled in the art should understand that the scope of disclosure involved in the present disclosure is not limited to the technical solutions formed by specific combinations of the above technical features, and shall also cover other technical solutions formed by any combination of the above technical features or equivalent features thereof without departing from the foregoing concept of disclosure. For example, a technical solution formed by a replacement of the above features with technical features with similar functions disclosed in the present disclosure (but not limited thereto) also falls within the scope of the present disclosure.

In addition, although the various operations are depicted in a specific order, it should be understood as requiring these operations to be performed in the specific order shown or in a sequential order. Under specific circumstances, multitasking and parallel processing may be advantageous. Similarly, although several specific implementation details are included in the foregoing discussions, these details should not be construed as limiting the scope of the present disclosure. Some features that are described in the context of separate embodiments may alternatively be implemented in combination in a single embodiment. In contrast, various features described in the context of a single embodiment may alternatively be implemented in a plurality of embodiments individually or in any suitable subcombination.

Although the subject matter has been described in a language specific to structural features and/or logical actions of the method, it should be understood that the subject matter defined in the appended claims is not necessarily limited to the specific features or actions described above. In contrast, the specific features and actions described above are merely exemplary forms of implementing the claims.

For the present disclosure, the following several points further need to be noted.

(1) The accompanying drawings of the embodiments of the present disclosure relate only to structures involved in the embodiments of the present disclosure, and for other structures, reference may be made to general designs.

(2) The embodiments of the present disclosure and features in the embodiments may be combined with each other without conflict, to obtain new embodiments.

The above description illustrates merely specific implementations of the present disclosure, but is not intended to limit the scope of protection of the present disclosure. The scope of protection of the present disclosure shall be subject to the scope of protection of the claims.

What is claimed is:

1. A vector generation method, comprising:
performing learnable processing on N current position vectors in a mapping table to obtain N learnable position vectors, wherein N is a positive integer; and
updating the N current position vectors with the N learnable position vectors.

2. The vector generation method according to claim 1, further comprising:
performing the learnable processing on N updated current position vectors again.

3. The vector generation method according to claim 1, wherein the updating the N current position vectors with the N learnable position vectors comprises:
replacing values of the N current position vectors in the mapping table with values of the N learnable position vectors, respectively.

4. The vector generation method according to claim 1, wherein the mapping table indicates a mapping relationship between a position parameter and a current position vector, and further comprises N position parameters in a one-to-one correspondence with the N current position vectors; and
the learnable processing comprises:
determining at least one position parameter set, wherein each position parameter set comprises at least one of the N position parameters;
obtaining at least one position vector set respectively corresponding to the at least one position parameter set based on the mapping table, wherein each position vector set comprises a current position vector that is obtained based on the mapping table and that corresponds to a position parameter in a position parameter set corresponding to the position vector set; and
processing the at least one position vector set to obtain the N learnable position vectors.

5. The vector generation method according to claim 4, wherein the processing the at least one position vector set to obtain the N learnable position vectors comprises:
separately processing the at least one position vector set using a neural network to obtain at least one output result respectively corresponding to the at least one position vector set;
calculating at least one loss value using a loss function corresponding to the neural network based on the mapping table, the at least one position parameter set, and the at least one output result; and
in response to the at least one loss value not satisfying a predetermined condition, correcting the N current position vectors based on the at least one loss value to obtain the N learnable position vectors; or
in response to the at least one loss value satisfying the predetermined condition, using the N current position vectors as the N learnable position vectors.

6. The vector generation method according to claim 5, wherein the calculating at least one loss value using a loss function corresponding to the neural network based on the mapping table, the at least one position parameter set, and the at least one output result comprises:
for each position parameter set:
determining a target result corresponding to the position parameter set based on the position parameter set;
obtaining the N current position vectors in the mapping table; and
calculating one loss value using the loss function corresponding to the neural network based on the N current position vectors, the target result, and an output result corresponding to a position vector set corresponding to the position parameter set.

7. The vector generation method according to claim 5, further comprising:
correcting a parameter of the neural network based on the at least one loss value,
wherein the loss function comprises an error loss function and a smoothness loss function.

8. (canceled)

9. The vector generation method according to claim 7, wherein N is greater than 1, and the smoothness loss function is expressed as:
$${L}_{smooth} = \frac{{\sum }_{i = 1}^{N - 1} ‖ h ( {x}_{i + 1} ) - h ( {x}_{i} ) ‖}{{\sum }_{i = 1}^{N - 1} ‖ h ( {x}_{i} ) ‖}$$
wherein Lₛₘₒₒₜₕ represents the smoothness loss function, ∥*∥ represents a 2-norm, Σ represents summation, h(xᵢ₊₁) represents an (i+1)th current position vector in the N current position vectors, h(xᵢ) represents an ith current position vector in the N current position vectors, and i is a positive integer and is less than or equal to (N−1);
wherein the error loss function comprises a root mean square error loss function or a mean absolute error loss function.

10. (canceled)

11. The vector generation method according to claim 7, wherein the loss function is expressed as:
$$L = {L}_{e} + λ * {L}_{smooth}$$
wherein L represents the loss function, Lₑ represents the error loss function, Lₛₘₒₒₜₕ represents the smoothness loss function, and χ represents a hyperparameter for controlling a contribution of the smoothness loss function and is a constant.

12. The vector generation method according to claim 4, wherein the obtaining at least one position vector set respectively corresponding to the at least one position parameter set based on the mapping table comprises:
for each position parameter in each position parameter set:
in response to the position parameter being different from any one of the N position parameters:
determining a first position parameter to be interpolated and a second position parameter to be interpolated that correspond to the position parameter, wherein the first position parameter to be interpolated and the second position parameter to be interpolated are two of the N position parameters, and the position parameter is located between the first position parameter to be interpolated and the second position parameter to be interpolated;
determining, based on the mapping table, a current position vector corresponding to the first position parameter to be interpolated as a first position vector to be interpolated and a current position vector corresponding to the second position parameter to be interpolated as a second position vector to be interpolated; and
performing interpolation on the first position vector to be interpolated and the second position vector to be interpolated to obtain a current position vector corresponding to the position parameter; or
in response to the position parameter being one of the N position parameters:
determining a current position vector corresponding to the position parameter directly based on the mapping table;
wherein an interpolation method corresponding to the interpolation comprises linear interpolation and/or cubic Hermite interpolation.

13. (canceled)

14. The vector generation method according to claim 4, wherein the determining at least one position parameter set comprises:
determining at least one piece of data to be processed; and
determining, based on the at least one piece of data to be processed, the at least one position parameter set in a one-to-one correspondence with the at least one piece of data to be processed;
wherein each piece of data to be processed corresponds to one molecule.

15. (canceled)

16. The vector generation method according to claim 4, further comprising:
determining a position parameter range based on a training dataset, wherein the training dataset comprises a plurality of pieces of training data; and
determining the N position parameters based on the position parameter range.

17. The vector generation method according to claim 16, wherein the determining a position parameter range based on a training dataset comprises:
determining a plurality of training position parameters based on the plurality of pieces of training data;
determining a first training position parameter and a second training position parameter in the plurality of training position parameters, wherein in the plurality of training position parameters, the first training position parameter is the largest, and the second training position parameter is the smallest; and
determining the position parameter range based on the first training position parameter and the second training position parameter;
wherein the N position parameters are evenly distributed in the position parameter range.

18. (canceled)

19. The vector generation method according to claim 16, wherein the determining a position parameter range based on a training dataset comprises:
determining a plurality of training position parameters based on the plurality of pieces of training data;
determining a first training position parameter and a second training position parameter in the plurality of training position parameters, wherein in the plurality of training position parameters, the first training position parameter is the largest, and the second training position parameter is the smallest;
separately performing transformation on the first training position parameter and the second training position parameter to obtain a first transformed training position parameter and a second transformed training position parameter; and
determining the position parameter range based on the first transformed training position parameter and the second transformed training position parameter;
wherein the transformation is performed on the N position parameters to obtain N transformed position parameters, and the N transformed position parameters are evenly distributed in the position parameter range.

20. (canceled)

21. The vector generation method according to claim 4, wherein each position parameter in the mapping table comprises a spacing between two adjacent atoms in a molecule and/or a bond angle in the molecule.

22. A data processing method, comprising:
obtaining a position parameter to be processed;
determining, based on a mapping table corresponding to the position parameter to be processed, a position vector to be processed that corresponds to the position parameter to be processed, wherein the mapping table comprises N current position vectors, and the N current position vectors in the mapping table are obtained through updating based on the N learnable position vectors obtained according to a vector generation method; and
processing, by using a neural network corresponding to the mapping table, the position vector to be processed to obtain a processing result,
wherein the vector generation method comprises:
performing learnable processing on N current position vectors in a mapping table to obtain N learnable position vectors, wherein N is a positive integer; and
updating the N current position vectors with the N learnable position vectors.

23. The data processing method according to claim 22, wherein the position parameter to be processed comprises a spacing between two adjacent atoms in a molecule and/or a bond angle in the molecule; and
the processing result comprises a predicted value of a property of the molecule.

24. A vector generation apparatus, comprising:
one or more memories storing computer-executable instructions in a non-transitory manner; and
one or more processors configured to run the computer-executable instructions,
wherein when the computer-executable instructions are run by the one or more processors, a vector generation method is implemented, and the vector generation method comprises:
performing learnable processing on N current position vectors in a mapping table to obtain N learnable position vectors, wherein N is a positive integer; and
updating the N current position vectors with the N learnable position vectors.

25. A data processing apparatus, comprising:
one or more memories storing computer-executable instructions in a non-transitory manner; and
one or more processors configured to run the computer-executable instructions,
wherein when the computer-executable instructions are run by the one or more processors, the data processing method according to claim 22 is implemented.

26. A non-transitory computer-readable storage medium, wherein the non-transitory computer-readable storage medium stores computer-executable instructions, and when the computer-executable instructions are executed by a processor, a vector generation method is implemented, or the data processing method according to claim 22 is implemented; and
the vector generation method comprises:
performing learnable processing on N current position vectors in a mapping table to obtain N learnable position vectors, wherein N is a positive integer; and
updating the N current position vectors with the N learnable position vectors.
